(19) United States
(12) Patent Application Publication
(10) Pub. No.: US 2019/0070096 A1
(43) Pub. Date: Mar. 7, 2019

(54) FOLLOWER COMPOSITION FOR COSMETIC APPLICATOR

(71) Applicant: MITSUBISHI PENCIL COMPANY, LIMITED, Shinagawa-ku, Tokyo (JP)

(72) Inventors: Keiichiro TAKACHIYO, Fujioka-shi, Gunma (JP); Satoshi SAKUMA, Fujioka-shi, Gunma (JP); Takayuki HAYAKAWA, Fujioka-shi, Gunma (JP)

(73) Assignee: MITSUBISHI PENCIL COMPANY, LIMITED, Shinagawa-ku, Tokyo (JP)

(21) Appl. No.: 16/084,543

(22) PCT Filed: Feb. 10, 2017
(86) PCT No.: PCT/JP2017/004994

§ 371 Date: Sep. 12, 2018

(30) Foreign Application Priority Data
Mar. 15, 2016 (JP) 2016-051082

(51) Int. Cl. A61K 8/90; A61K 8/31; A61Q 3/00; A61Q 1/00

(52) U.S. Cl. CPC A61K 8/90; A61K 8/31; A61K 2800/10; A61Q 1/00; A61K 2800/87; A61Q 3/00

(57) ABSTRACT

Provided is a follower composition for a cosmetic applicator contributing to stable discharge, excellent holding property for aging stability, and easy to find the end of a liquid cosmetic regardless of any visibility of filling chamber and any type of coating part. The follower composition for a cosmetic applicator is characterized by comprising at least a base oil and 3.5 to 8% by mass of a styrene copolymer elastomer. The follower composition for a cosmetic applicator is used as a follower which is brought into contact with a liquid cosmetic and which moves following the liquid cosmetic reduced as it is consumed in a cosmetic applicator comprising, for example, a cylindrical filling chamber, communicating directly or via an interposition member with a coating part to receive the liquid cosmetic directly.

### TECHNICAL FIELD

The present invention relates to a follower composition (follower) for a cosmetic applicator which contributes to stable discharge of a liquid cosmetic and is excellent in an aging stability holding property and which is easy to find the end of using the liquid cosmetic regardless of whether or not a filling chamber for filling the liquid cosmetic is visible and whether or not an applying part is a ballpoint pen type.

### BACKGROUND ART

A device for redispersing a pigment has so far conventionally been indispensable in an applicator in which a cosmetic having a low viscosity and containing a pigment is filled and held. That is, it is a device which is put in a filling chamber for a the cosmetic together with a stirrer such as a stirring ball or a stirring rod to shake the whole part of the applicator until the stirrer sounds like moving around sufficiently when the pigment contained therein is estimated to be settled down after not used for a long time to make it possible to redisperse the cosmetic (refer to, for example, a patent document 1).

Also, the applicator described above is provided with an applying tip of a so-called ballpoint pen type in which a ball is continuously pushed forward to a ball holder, and it is constituted so that when the pushing is released, the liquid cosmetic stored in the filling chamber comes out by substitution with air. This system involves the problems that when the cosmetic has a low viscosity, the cosmetic oozes out from the tip of the applicator and that when the pushing is released, an unexpectedly large amount of the cosmetic comes out.

An applicator of a mechanism where in place of a mechanism in which the cosmetic is discharged by substitution with air in the applicator described above, a ballpoint pen tip comprising a ball and a ball holder, a cosmetic and a stirrer in a filling chamber and a liquid follower at a rear part thereof are provided at a tip of a cylindrical filling chamber and in which a space formed by a portion of the cosmetic oozed out is made up by the movement of the follower is disclosed by the present applicants (refer to, for example, a patent document 2). In the applicator of the mechanism described above, the stirring ball is likely to be sunk into the soft follower when shaking the applicator, and therefore a cushioning member is provided between the cosmetic and the follower to prevent the stirring ball from being sunk into the follower.

Further, disclosed as well is an applicator provided with a mechanism close to that of a so-called gel ink ballpoint pen in which a cosmetic itself is gelatinized (provided with a structural viscosity) without providing a stirrer to require no stirring and in which a follower is provided (refer to, for example, a patent document 3). In the applicator of the mechanism described above, a float (follower auxiliary member) is inserted from a rear side of the follower in order to endure a falling impact when the applicator falls from a rear side, and the problem that when such an accident that the float is brought into contact with a wall surface of the cylindrical filling chamber is caused, the air comes in to lead to bringing about an anxiety in an aging stability.

### RELATED ART DOCUMENTS

### Patent Documents

Patent document 1: Japanese Patent Application Laid-Open No. Hei. 5-7822 (claims, examples, FIG. 1 and the like)
Patent document 2: Japanese Patent Application Laid-Open No. 2007-272 (claims, examples, FIG. 1 and the like)
Patent document 3: Japanese Patent Application Laid-Open No. 2001-340131 (claims, examples, FIG. 1 and the like)

### DISCLOSURE OF THE INVENTION

### Problems to be Solved by the Invention

The present invention intends to solve such the problems on the conventional technologies as described, and an object thereof is to provide, while succeeding the excellent matters of the conventional technologies, a follower composition for a liquid cosmetic which contributes to stable discharge of the liquid cosmetic and is excellent in an aging stability holding property and which is easy to find the end of using the liquid cosmetic regardless of whether or not a filling chamber for filling the liquid cosmetic is visible and whether or not an applying part is a ballpoint pen type.

### Means for Solving the Problem

In light of the conventional problems described above and the like, intense investigations repeated by the present inventors have resulted in finding that a follower composition for a liquid cosmetic which is used for a cosmetic applicator filled with a liquid cosmetic and which moves following the liquid cosmetic reduced as it is consumed is obtained by using a follower composition (follower) having specific physical properties as a result of investigating a blend composition. Thus, they have come to complete the present invention.

That is, the present invention resides in the following items (1) to (4).

(1) A follower composition for a cosmetic applicator comprising at least a base oil and 3.5 to 8% by mass of an elastomer having a styrene structure.
(2) The follower composition for a cosmetic applicator as described in the above item (1), wherein the elastomer having a styrene structure comprises a styrene-ethylene/propylene block copolymer.
(3) The follower composition for a cosmetic applicator as described in the above item (1) or (2), wherein the base oil is a liquid paraffin.
(4) The follower composition for a cosmetic applicator as described in any one of the above items (1) to (3), wherein an average value of tan δ in a frequency range of 1 to 20 Hz is 0.25 to 0.45.

### Effects of the Invention

According to the present invention, provided is a cosmetic applicator which contributes to stable discharge of the liquid cosmetic and is excellent in an aging stability holding property and which is easy to find the end of using the liquid cosmetic regardless of whether or not a filling chamber for filling a liquid cosmetic is visible and whether or not an applying part is a ballpoint pen type.

### BRIEF DESCRIPTION OF THE DRAWINGS

FIG. 1a and FIG. 1b show one example of the embodiment of using the follower composition for a cosmetic applicator according to the present invention, wherein (a) is a front view, and (b) is a vertical cross-sectional view

FIG. 2a and FIG. 2b show a state in which the cap of the cosmetic applicator shown in FIG. 1a and FIG. 1b is removed, wherein (a) is a front view, and (b) is a vertical cross-sectional view.

FIG. 3a and FIG. 3b show another example of the embodiment of using the follower composition for a cosmetic applicator according to the present invention, wherein (a) is a vertical cross-sectional view in closing the valve, and (b) is a vertical cross-sectional view in opening the valve.

### MODE FOR CARRYING OUT THE INVENTION

The follower composition for a cosmetic applicator according to the present invention shall be explained below in detail.

The follower composition for a cosmetic applicator according to the present invention is characterized in comprising at least a base oil and 3.5 to 8% by mass of an elastomer having a styrene structure.

The follower composition for a cosmetic applicator used in the present invention is constituted by at least one selected from liquid gelled substances containing at least a base oil and an elastomer having a styrene structure in terms of maintaining the quality thereof to a high extent without losing a function as an aqueous liquid cosmetic.

The liquid gelled substance containing at least the base oil and the elastomer having a styrene structure is constituted preferably by, for example, a substance containing at least a non-volatile or scarcely volatile base oil and an elastomer having a styrene structure.

At least one (one kind or two or more kinds, hereinafter the same shall apply) selected from, for example, a liquid paraffin, a mineral oil, squalane and the like can be used as the usable non-volatile or scarcely volatile base oil described above.

The base oils such as the liquid paraffin, mineral oil, squalane and the like described above are used as blend components for cosmetics such as manicure compositions (nail liquids), eye shadow liquids, cosmetics for foundation, eyeliner liquids and the like, and they are excellent in a stability and a safety.

The base oils such as liquid paraffins, mineral oils, squalane and the like which can be used shall not specifically be restricted as long as they are used as blend components for cosmetics, and commercial products can be used. The liquid paraffins out of the base oils described above are preferably used in terms of a cost, a fluidity and the like.

The elastomer having a styrene structure used in the present invention is soluble swollen in the non-volatile or scarcely volatile base oil described above, and it may be any compounds as long as it can turn the non-volatile or scarcely volatile base oil into a gelled substance. It includes, for example, at least one selected from styrene base thermoplastic elastomers.

The styrene base thermoplastic elastomers which can be used include materials in which at least polystyrene constitutes a hard segment (x) and in which the other polymer (for example, polybutadiene, polyisoprene, polyethylene, hydrogenated polybutadiene, hydrogenated polyisoprene and the like) is a soft segment (y) which is amorphous and has a low glass transition temperature.

The combination of the hard segment and the soft segment described above can include the respective combinations of polystyrene with polybutadiene, polyisoprene, polyethylene, hydrogenated polybutadiene, hydrogenated polyisoprene and the like, and among them, the combination of polystyrene/polybutadiene and the combination of polystyrene/polyisoprene are preferred. Also, in order to inhibit the unexpected cross-linking reaction, the soft segment is preferably hydrogenated. The ratio of the respective combinations described above shall not specifically be restricted, and it is preferably x:y=5 to 95:95 to 5, preferably 10 to 40:90 to 60 in terms of a mass ratio.

The polystyrene base thermoplastic elastomer which can be used can be synthesized by copolymerizing a polymer constituting the hard segment with a polymer constituting the soft segment by a publicly known method and includes, for example, styrene-butadiene base copolymers (copolymers): SBS (polystyrene-poly(butylene) block-polystyrene), SEBS (polystyrene-poly(ethylene/butylene) block-polystyrene), styrene-isoprene copolymer: styrene-isoprene block-styrene), styrene-propylene base copolymer: SEP (polystyrene-(ethylene/propylene) block, SEPS (polystyrene-poly(ethylene/propylene) block-polystyrene), SEEPS (polystyrene-poly(ethylene-ethylene/propylene) block-polystyrene), SEB (polystyrene-(ethylene/butylene) block), styrene-isobutylene-styrene block copolymer (SIBS), and the like.

Among the polystyrene base thermoplastic elastomers described above, the styrene-ethylene/propylene) block copolymer is preferably used in terms of a viscoelasticity and a defoaming property.

Commercial products such as, for example, a styrene elastomer G1701EU, G1702HU manufactured by Kraton Polymer Japan Corp., Rabalon SJ4300C, Rabalon SJ5300C, Rabalon SJ6300C and the like manufactured by Mitsubishi Chemical Corporation can be used as the polystyrene base thermoplastic elastomers described above.

A content of the above elastomers having a styrene structure has to be 3.5 to 8% by mass based on a whole amount of the follower composition for a cosmetic applicator.

If the content is less than 3.5 by mass and exceeds 8% by mass, the liquid cosmetic is damaged in a stability of discharge and an aging stability of the liquid cosmetic in a certain case, and the effects of the present invention cannot be exerted.

Also, a content of the usable non-volatile or scarcely volatile base oil described above is 70 to 96.5% by mass, preferably 85 to 96.5% by mass and more preferably 92 to 96.5% by mass based on a whole amount of the follower composition for a cosmetic applicator.

Further, the follower composition for a cosmetic applicator according to the present invention has a specific gravity of preferably less than 100%, particularly preferably 90 to 30% based on a specific gravity of the liquid cosmetic used in terms of an efficient followability.

In the present invention, the follower composition for a cosmetic applicator is changed in a specific gravity depending on the kind of the aqueous liquid cosmetics used (manicure compositions (nail liquids), eye shadow liquid ds, eyebrow liquids, cosmetics for foundation, liquid hair dyes, eyeliner liquids, cosmetics for lips), and the concentrations thereof. For example, when a specific gravity of the manicure composition (nail liquid) is 1.2, a specific gravity of the insoluble or scarcely soluble follower composition for a cosmetic applicator is preferably less than 100% (specific gravity: 1), more preferably 90 to 30% (0.9 to 0.3).

In the present invention, the liquid cosmetic used is varied in a specific gravity according to the uses and the blend kind thereof, and therefore the follower composition for a cosmetic applicator having the specific gravity range described above can be prepared by suitably combining the kind and the use amount of the insoluble or scarcely soluble base oil, the elastomer having a styrene structure and a use amount thereof, and a manufacturing method thereof.

Further, an average value of a tan δ of the follower composition for a cosmetic applicator according to the present invention in a frequency range of 1 to 20 Hz (6.3 to 125.6 rad/sec) is preferably 0.15 to 0.45, more preferably 0.25 to 0.45 in terms of a convenience in production or an efficient followability, and the average value of the tan δ in the range described above is particularly preferably 0.25 to 0.35.

When the follower in which the tan δ is over 0.45 in the specific frequency range between 1 to 20 Hz is used for a cosmetic consumed in a particularly large amount, brought about are the problems that blurring is caused in the middle of use due to a difficulty in following, that a part of the follower is adhered to an inner wall of the filling chamber and remains thereon to result in being sort of the follower and causing countercurrent of the cosmetic and that the cosmetic is instable in a flow amount due to influence of the short follower. Also, even in a case where the cosmetic is not used in a large consumption amount, the same problems as described above are caused when a consumption speed is raised.

Further, if the tan δ is over 0.45 in all the frequency range between 1 to 20 Hz, brought about are the problems that the follower is inferior in a following responsiveness in consuming the cosmetic and that when an impact is applied to the housing part, the follower is liable to be scattered.

On the contrary, if the tan δ is below 0.15 in the specific frequency range or all the frequency range between 1 to 20 Hz, the elasticity of the follower is considerably strong, so that the follower is difficult to be charged in the filling chamber and loses a practicality.

In the present invention, the volume of the follower composition for a cosmetic applicator having the constitution described above is preferably 0.5 or more, more preferably 0.5 to 3 assuming that the volume of the liquid cosmetic is 1 in terms of a good followability and an impact resistance in falling.

Also, the follower composition for a cosmetic applicator having the constitution described above has preferably a hue different from that of the liquid cosmetic in terms of distinct identification from the liquid cosmetic; for example, if the liquid cosmetic is colored, the follower composition for a cosmetic applicator is colorless; if the liquid cosmetic is colorless, the follower composition for a cosmetic applicator is colored (black, yellow and the like) by adding a suitable amount of an oil-soluble dye used for cosmetic materials.

The follower composition for a cosmetic applicator thus constituted according to the present invention is used for, for example, a cosmetic applicator prepared by filling a liquid cosmetic.

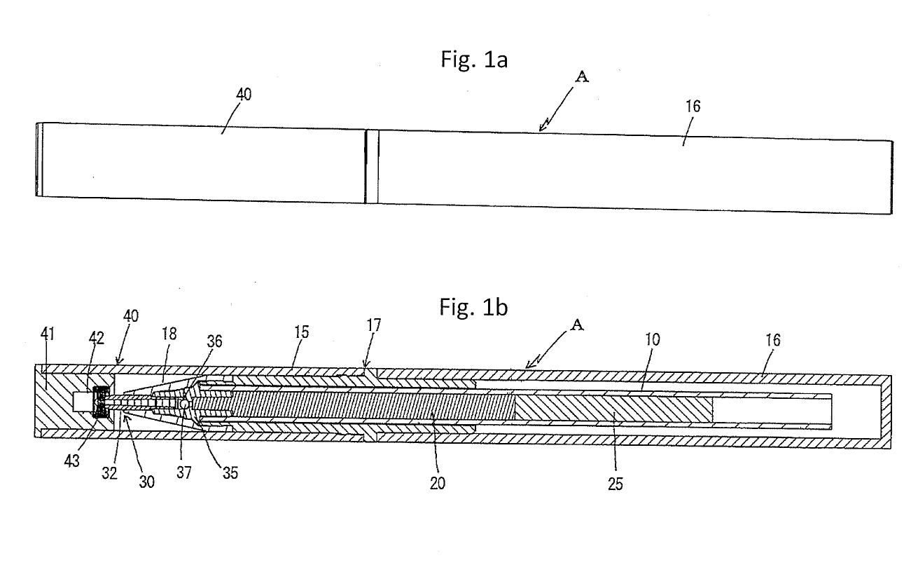

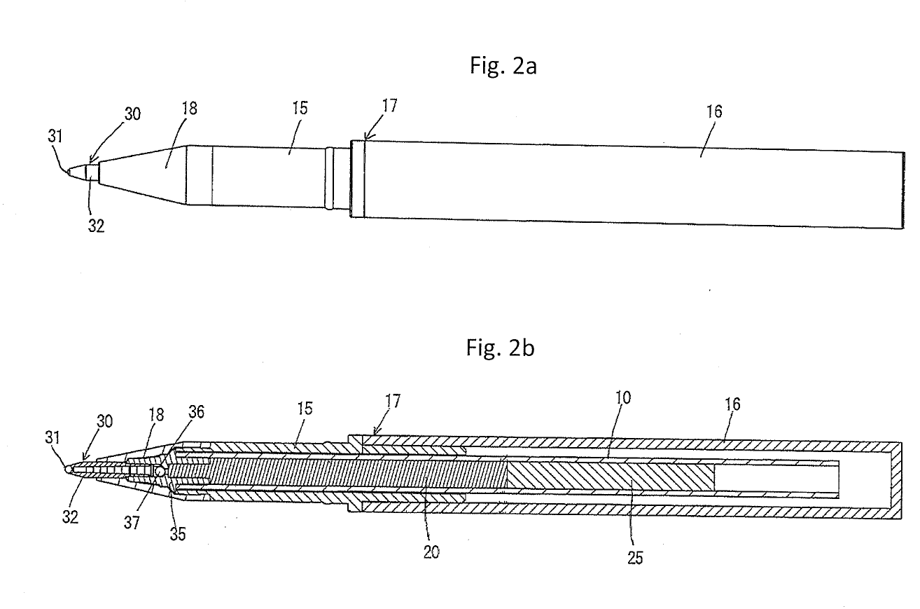

FIGS. 1a and 1b and FIGS. 2a and 2b are drawings showing one example of using the follower composition for a cosmetic applicator according to the present invention (hereinafter referred to as the follower).

As shown in FIGS. 1a and 1b, and FIGS. 2a and 2b, a cosmetic applicator A in the present embodiment comprises a refill type cylindrical filling chamber 10 in which a liquid cosmetic 20 is directly filled, and an applying part 30 constituted by a ball holder for holding an applying ball is mounted at the tip of the cylindrical filling chamber 10.

The cosmetic applicator A is constituted so that a cylindrical front shaft 15 opened in a front part and a rear part and a bottomed cylindrical rear shaft 16 are fixed by fixing means such as screwing, pressing and the like to constitute a holder 17 and that the refill type cylindrical filling chamber 10 is housed in the holder 17 by a plastic tip 18 screwed into the tip of the holder 17. In the present embodiment, the plastic tip 18 is not necessarily required, and it may be a part integrated with the front shaft 15. A vent hole is drilled in a suitable position communicating into an inside of the holder 17, or a groove is installed in a screwing part in which the plastic tip 18 is fixed, whereby the holder 17 is provide with the vent hole or the vent groove. A vent hole communicating into an inside of the holder can be installed at rear end of the rear shaft 16 constituting the holder 17 (not illustrated). A method for fixing the plastic tip 18 shall not necessarily restricted to screwing and may be fixing by pressing. Also, a number 40 shows a cap freely attachable by fitting the front shaft 15.

The refill type cylindrical filling chamber 10 and the holder 17 constituted by the front shaft 15 and the bottomed cylindrical rear shaft 16 are constituted by metals such as aluminum and stainless, synthetic resins, glass and the like, and they are formed preferably by integration molding by using resinous materials such as polypropylene, polyvinyl alcohol, ethylene-vinyl alcohol copolymer resin (EVOH), polyacrylonitrile, nylon, cellophane, polyethylene terephthalate, polycarbonate, polystyrene, polyvinylidene chloride, polyvinyl chloride, and the like.

More preferably, the refill type cylindrical filling chamber 10 and the holder 17 constituted by the front shaft 15 and the bottomed cylindrical rear shaft 16 are formed preferably by materials having a light transmittance of 50% or more, and materials having a light transmittance of 80% or more are particularly preferred in order to make it easier to recognize (liable to visually confirm) the end of consuming the liquid cosmetic. In the present invention, the “light transmittance” means a visible light transmittance.

An inner wall surface of the cylindrical filling chamber 10 described above, that is, a wall surface with which the liquid cosmetic is brought into contact may be subjected to water repellent film forming treatment by coating using a water repellent agent of a silicone base, a silicon resin base or a fluorine base. The wall surface with which the liquid cosmetic is brought into contact is subjected to water repellent film forming treatment in which it is controlled to a lower surface free energy than that of the liquid cosmetic, whereby it can be improved in a water repellency and set so that the end of consuming the liquid cosmetic is easier to recognize.

The liquid cosmetic 20 stored in the filling chamber 10 shall not specifically be restricted as long as it is a cosmetic containing water or water and a water-soluble solvent as a main solvent (hereinafter referred to as an “aqueous” cosmetic), and it includes manicure compositions (nail liquids), eye shadow liquids, eyebrow liquids, cosmetics for foundation, liquid hair dyes, eyeliner liquids, cosmetics for lips, and the like. Also, the liquid cosmetic 20 is varied in a viscosity according to the kind thereof and is suitably 500 to 3000 (m·Pa·s) measured at a measuring temperature of 25° C. by means of a cone plate type viscometer.

In the present embodiment, a manicure composition (nail liquid, white color) is housed as the liquid cosmetic 20 in the filling chamber 10.

A follower 25 which is brought into tight contact with an inner wall surface of the filling chamber 10 while brought into contact with the liquid cosmetic 20 and which moves following the liquid cosmetic 20 reduced as it is consumed is mounted in a rear end part of the liquid cosmetic 20 described above, wherein the liquid follower comprises the blend composition described above having a hue different from that of the liquid cosmetic 20. In the present embodiment, the follower 25 is constituted from liquid paraffin and an elastomer (color: colorless) having a styrene structure, and a volume of the follower is 0.63 assuming that a volume of the liquid cosmetic described above is 1.

In the cosmetic applicator A of the present embodiment, the plastic tip 18 is formed by metal and assumes a conical form in which front and rear ends are opened, and an annular step part in which a diameter is decreased toward a front part is formed on an inner peripheral surface.

The applying part 30 comprises a metal pipe type (needle type) ballpoint pen tip 32 having an applying ball 31 at a tip, wherein a tip side is fixed by pressing in the plastic tip 18, and a rear end side is fixed by pressing in a joint member 35 which causes the aqueous liquid cosmetic stored in the filling chamber 10 to flow into the ballpoint pen tip 32 of the applying part 30.

The joint member 35 is provided in an inside with a valve chamber 36, and a ball valve 37 is installed in the valve chamber 36; and in an applying state in which the ballpoint pen tip 32 is turned downward, the ball valve 37 is brought into contact with a front side of the joint member 35, and a gap is formed in a rear end part of the joint member 35 to allow the liquid cosmetic to flow into the ballpoint pen tip 32. Also, in a state in which the ballpoint pen tip 32 is turned upward, the ball valve 37 is brought into contact with a rear end side which is a ball receiving seat side of the joint member 35 to tightly close a flow passage of the liquid cosmetic, and therefore the cosmetic applicator A is so constituted that if coated with the ballpoint pen tip turned upward, the liquid cosmetic does not move to a rear side of the refill and therefore is prevented from causing countercurrent. Also, a sealing part 41 is fixed in the cap 40 described above, and a sealing plug body 43 for sealing the tip of the applying ball 31 is provided in a concave part 42 of the sealing part 41.

In the cosmetic applicator A of the present embodiment, outside air is introduced through a vent hole (not illustrated) arranged in a predetermined position of the holder 17 when the aqueous liquid follower 25 moves, so that the liquid follower 25 is prevented from blocking the movement due to a reduction in an inner pressure of a space part of the filling chamber 10, and the liquid follower 25 follows smoothly the consumption of the aqueous liquid cosmetic 20. Accordingly, the liquid cosmetic 20 is smoothly discharged from the applying ball 31 of the applying part 30, and a rear end part of the aqueous liquid cosmetic 20 is provided with a structure in which it is sealed by the liquid follower 25, so that the aqueous liquid cosmetic 20 is provided with such a constitution as excellent in an aging stability.

The cosmetic applicator A thus constituted in the present embodiment comprises the cylindrical filling chamber 10, the applying part 30 installed at the tip of the cylindrical filling chamber 10, the aqueous liquid cosmetic 20 filled directly in the cylindrical filling chamber 10 communicating with the applying part 30 via the joint member 35 having the ball valve 37 which is an interposition member, and the follower 25 of the present invention which is brought into tight contact with the liquid cosmetic 20 and which moves following the liquid cosmetic 20 reduced as it is consumed and has a hue different from that of the liquid cosmetic 20, wherein the follower 25 contains at least a base oil and 3.5 to 8% by mass of an elastomer having a styrene structure. According to the constitution described above, provided is follower which contributes to a stability of discharging the liquid cosmetic 20; which is excellent in a holding property for an aging stability of the liquid cosmetic 20; the filling chamber 10 and the holder 17 are excellent in a visibility, and therefore if the light transmittance is controlled to 80% or more, they are more excellent in a visibility; and the end of consuming the aqueous liquid cosmetic 20 is more easily recognized.

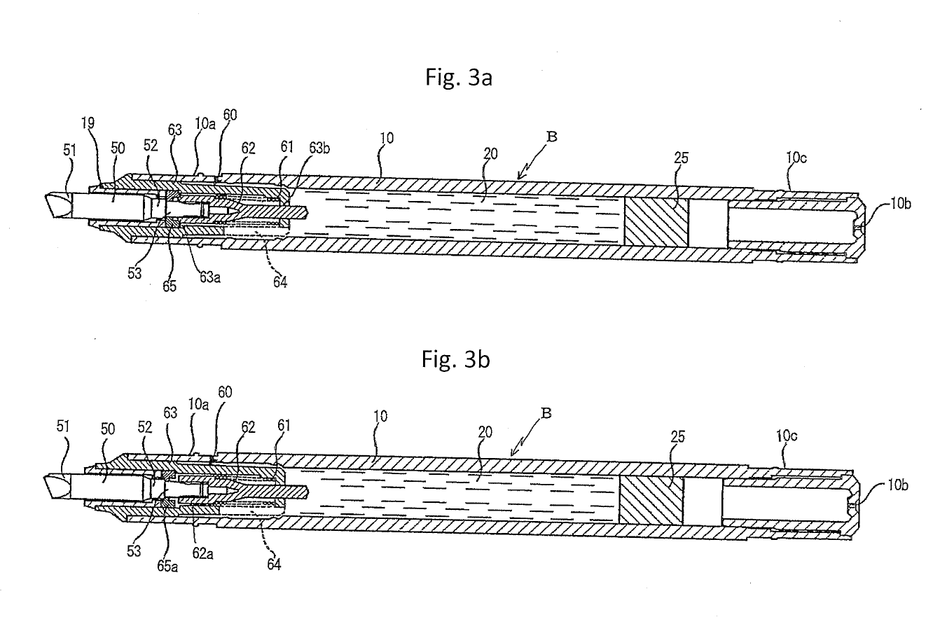

FIGS. 3a and 3b show another example of the embodiment of using the follower composition for a cosmetic applicator according to the present invention, wherein FIG. 3a is a vertical cross-sectional view in closing the valve, and FIG. 3b is a vertical cross-sectional view in opening the valve. The explanations of the parts and the like in the same constitution as described above are omitted because of the same codes.

The cosmetic applicator B of the present embodiment comprises, as shown in FIG. 3a and FIG. 3b, a cylindrical filling chamber 10 which is a holder (shaft body) grasped by the user and constituted in the same mode as in the embodiment described above and in which a liquid cosmetic 20 is directly filled.

An applying part 50 is installed at a tip of the cylindrical filling chamber 10 described above. In the present embodiment, the applying part 50 of a pen feed type in which a front part 51 is protruded from a tip part 10a of the cylindrical filling chamber 10 is installed, and a valve mechanism part 60 as an interposition member in which a valve is closed by pressing of an elastic member 61 comprising a coil spring in applying no force to the applying part 50 and in which the valve is opened in applying an external force to introduce a liquid cosmetic 20 stored in the filling chamber 10 into the applying part 50 is mounted at a rear side of the applying part 50.

A follower 25 having a hue different from that of the liquid cosmetic 20 and constituted in the same manner as in the embodiment described above is brought into tight contact with an inner wall surface of the filling chamber 10 while brought into contact with the liquid cosmetic 20 and moves following the liquid cosmetic 20 reduced as it is consumed is mounted in a rear end part of the aqueous liquid cosmetic 20 described above.

In the cosmetic applicator B of the present embodiment, a rear end part of a filling chamber 10 is blocked by a tail plug 10c in which a vent hole 10b is formed, and outside air is introduced through the vent hole 10b when the liquid follower 25 moves, so that the liquid follower 25 is not prevented from blocking the movement due to a reduction in an inner pressure of a space part of the filling chamber 10, and the follower 25 follows smoothly the consumption of the liquid cosmetic 20.

A hollow cylindrical guide member 19 so-called “kuchipura” which slidably guides an applying part 50 is arranged at front side of a valve mechanism part 60 in a tip part 10a of the filling chamber 10.

The valve mechanism part 60 allows the guide member 19 to be fitted into an inside at a front side and has a cylindrical support member 63 housing a valve rod 62 and an elastic member 61 pushing the valve rod 62 forward at a rear side of the guide member 19, and a communication hole 64 for communicating an inside of the filling chamber 10 and a valve body is formed in a rear end part of the cylindrical support member 63.

The applying part 50 is a resin-made or fiber-made molded lead, a fiber bundle lead, or a burned lead obtained by burning a resin or a fiber. The resin is preferably POM, PP, nylon, PE and the like, and the fiber or the fiber bundle is preferably PET, acryl, nylon, wool and the like. The applying part 50 is integrally molded by the materials described above, and in addition thereto, it can be provided with a structure in which a skin member such as a liquid-tight resin-made film or a cylindrical body is integrally adhered tightly around the periphery. The applying part 50 of the present embodiment is constituted by a PET-made fiber bundle.

The elastic member 61 is suitably a coil spring made of stainless, and in addition thereto, a shape and a material such as a plate, an elastomer and the like shall not be restricted as long as it can press the valve rod 62 forward.

In the valve mechanism part 60, an outer peripheral part of a valve body 65 is sandwiched between a rear end part surface of the guide member 19 and a diameter-reducing step part 63a having a smaller diameter toward a front side in the cylindrical support member 63, whereby the valve body 65 is fixed in an inside of the valve mechanism part 60. A rear part 52 having a smaller diameter than that of a front part 51 of the applying part 50 is inserted into a central hole 65a of the valve body 65.

An opening part is formed in a front part of the valve rod 62 to expand the diameter, and a rear part thereof is solid and formed in a small diameter. A small diameter part 53 at a rear part of the applying part 50 is inserted into an opening part 62a and tightly fixed. The valve mechanism part 60 allows the guide member 19 to be fitted into an inside at a front side and has the cylindrical support member 63 housing the valve rod 62 and the elastic member 61 pushing the valve rod 62 forward at a rear side of the guide member 19, and the communication hole 64 for communicating an inside of the filling chamber 10 and the valve rod 62 is formed in a rear end part of the cylindrical support member 63.

To be specific, the cylindrical support member 63 assumes roughly a container shape which is closed at a rear part approximately in a wall shape and is opened at a front part, and a small size part 53 at a rear part of the applying part 50 is mounted movably in front and rear directions in a through hole 63b formed at a rear end part. Communication holes 64 of suitable size and number are formed at a side surface part of the cylindrical support member 63.

The elastic member 61 is interposed between a step part on a peripheral surface of a large diameter front part 62a of the valve rod 62 and a wall part at a rear end of the cylindrical support member 63, and it is mounted in a position around the valve rod 62.

In this regard, the valve mechanism part 60 has a valve function in which the valve is opened and closed by bringing a front end part of the valve rod 62 connected to the small diameter part 53 at a rear side of the applying part 50 into contact with the valve body 65 positioned at a rear side of the applying part 50 and comprising a flexible rubber elastic body, or separating the front end part of the valve rod 62 from the valve body 65.

The valve body 65 is provided with a constitution in which an inner peripheral end part of the valve body 65 is adhered to an outside surface of the applying part 50 in a pressed state and in which the inner peripheral end part is deformed by the movement of the coating part 50 while maintaining the adhered state to block outside air flow between the outside surface of the applying part 50 and the filling chamber 10. The flexible valve body 65 is provided with a structure in which the flexible valve body 65 is fitted into a side of the applying part 50 and in which a rear end thereof is brought into contact with a front end of the valve rod 62 by pressing of the elastic member 61 comprising a coil spring to block the filling chamber 10 from the outside.

The applying part 50 goes backward by an applying pressure or a higher pressing pressing pressure, whereby the valve mechanism part 60 is opened in a valve, and at the same time, the flexible valve body 65 fitted into the applying part 50 slides as well together with the applying part 50 while deforming. When this allows the valve comprising the valve body 65 and the valve rod 62 to be opened, the liquid cosmetic 20 is brought into contact with the applying part 50, and the liquid cosmetic 20 is fed. Temporarily, the filling chamber 10 is reduced in a pressure, and air tries to come in from the outside, but the flexible valve body 65 fitted into the applying part 50 provides a constitution of preventing the air from coming in from the outside.

The cosmetic applicator B thus constituted in the present embodiment comprises the cylindrical filling chamber 10, the applying part 50 constituted by a fiber bundle lead or a sintered lead and installed at the tip of the cylindrical filling chamber 10, the aqueous liquid cosmetic 20 filled directly in the cylindrical filling chamber 10 communicating with the applying part 50 via the valve mechanism part 60 which is an interposition member, and the liquid follower 25 which is brought into tight contact with the liquid cosmetic 20 and which moves following the liquid cosmetic 20 reduced as it is consumed and has a hue different from that of the liquid cosmetic 20, wherein the volume of the follower is 0.5 or more assuming that the volume of the liquid cosmetic is 1. According to the constitution described above, provided is a cosmetic applicator which contributes to a stability of discharging the liquid cosmetic 20; which is excellent in a holding property for an aging stability of the aqueous liquid cosmetic 20; the filling chamber 10 and the holder 17 are excellent in a visibility, and therefore if the light transmittance is controlled to 80% or more, they are more excellent in a visibility; and therefore the end of consuming the aqueous liquid cosmetic 20 is more easily recognized.

The follower composition for a cosmetic applicator according to the present invention shall not be restricted to the embodiment described above and can be varied in the various manners and used as long as the scope of the present invention is not changed.

For example, the color of the follower was colorless in the embodiment described above, but a suitable amount of an oil-soluble dye may be added to provide the follower with other hues (black, red, blue, yellow and the like) as long as it is different from a hue of the aqueous liquid cosmetic and allowed in terms of a design.

Also, the ball holder holding a ball, the fiber bundle lead, the sintered lead and the like were used as the applying part mounted at the tip of the cylindrical filling chamber 10, but it may be a brush type elastomer applicator.

### EXAMPLES

Next, the present invention shall be explained in further details with reference to examples and comparative examples, but the present invention shall not be restricted by the following examples and the like.

### Examples 1 to 6 and Comparative Examples 1 and 6

The respective blend prescriptions shown in the following Table 1, to be specific, base oils A to C, elastomers A, B and a colorant were used to prepare follower compositions for a cosmetic applicator having the respective hues in the respective blend amounts shown in the following Table 1.

Further, the components were mixed and dispersed in the blend prescriptions shown below by means of a homo mixer or a disper to prepare manicure compositions (nail liquids, whole amount: 100% by mass).

Liquid Cosmetic Composition, Color: White:
Colorant: titanium oxide (CR-50, manufactured by Ishihara Sangyo Kaisha, Ltd.) 55% by mass
Coating resin, dispersant: octylacrylamide/acrylic ester copolymer (AMPHOMER HC aqueous solution, manufactured by Akzo Nobel N.V.) 10% by mass
Thickener: succinoglycan (RHEOZAN SH, manufactured by Solvay Nicca N.V.) 26% by mass
Preservative: ehtlhexyl glycerin (Sensiva SC50JP, manufactured by Shulke & Mayr UK Ltd.) 1% by mass
Defoaming agent: silicone base defoaming agent (KM-72, manufactured by Shin-Etsu Chemical Co., Ltd.) 1% by mass
Solvent: 1,3-butylene glycol 0.3% by mass
Preservative: ethylparabenne, methylparabenne (manufactured by Midori Kagaku Co., Ltd.) 0.4% by mass
Refined water: 6.3% by mass

The viscosity values thereof were 500 to 3000 (m·Pa·s) measured at 25° C. by means of a cone plate type viscometer. Also, the specific gravities of the respective follower compositions for a cosmetic applicator obtained above based on the resulting liquid cosmetic are shown in the following Table 1. Further, the respective tan δ at a frequency range of 1 to 20 Hz (6.3 to 125.6 rad/sec) were measured by the following method, and the respective tan δ at a frequency range of 1 to 20 Hz and the average values thereof are shown in the following Table 2.

### Measuring Method of Tan δ: Examples 1 to 6 and Comparative Examples 1 and 6

Measuring device: Dynamic Spectrometer RDS-II (manufactured by Rheometric Scientific, Inc.)

Measuring Conditions (Frequency Dependency):
Geometry: parallel plate 50 mm ϕ dynamic measurement SWEEP TYPE: FREQUENCY SWEEP
Frequency range: 0.1 to 20 Hz (0.63 to 125.6 rad/s)
Measuring interval: 6 points/decade
Strain: 100%
Measuring temperature: 25° C.
Atmosphere: in nitrogen flow

The liquid cosmetics and the respective follower compositions for a cosmetic applicator obtained above were used and filled into a cosmetic applicator shown in FIGS. 1a and 1b.
Coating ball 31: ϕ1 mm
Ballpoint pen tip 32: made of stainless steel
Filling chamber 10: made of polypropylene, inner diameter 4 mm, outer diameter 6 mm, length 96 mm, light transmittance 80%
Holder 17, front shat 15: made of polypropylene, light transmittance 80%
Front shaft 16: made of ABS, light transmittance 80%

The applicators obtained by using the respective follower compositions for a cosmetic applicator prepared in Examples 1 to 6 and Comparative Examples 1 and 6 were used to evaluate an aging stability, a usability, a followability, a dripping resistance, and a bubble discharge property by the following methods to subject them respectively to comprehensive evaluation. The results thereof are shown in the following Table 1.

Evaluation Method of Aging Stability:

The respective cosmetic applicators obtained were left standing upward and downward under the environment of 50° C. to evaluate an aging stability in the state after 1 month according to the following evaluation criteria.

Evaluation Criteria:

◯: no change or a little change in an appearance but no influence in a usability

Δ: large change in appearance and exerting an influence on a usability

x: no usable

Evaluation Method of Usability:

The respective cosmetics obtained were used on nails to evaluate a usability according to the following evaluation criteria.

Evaluation Criteria:

◯: could be coated only by touching lightly or could be coated only by touching

Δ: could be coated by rubbing lightly the applying part onto a nail

x: could not be coated without rubbing considerably the applying part onto a nail, or could not at all be coated

Evaluation Method of Followability:

The respective cosmetic applicators obtained were used to continuously write circles on four sheets of writing papers and then evaluate a followability according to the following evaluation criteria.

Evaluation Criteria:

◯: drawn lines were not at all broken or scarcely broken

Δ: drawn lines were broken frequently in the beginning and the middle of writing

x: could not be written in the middle

Evaluation Method of Dripping Resistance:

The respective cosmetic applicators obtained were left standing upward at 50° C. for one week and then used to evaluate a dripping resistance according to the following evaluation criteria.

Evaluation Criteria:

◯: no change or little change in a shape

Δ: a little change in the bulk and the follower

x: the follower flowed out

Evaluation Method of Bubble Discharge Property:

The respective cosmetic applicators obtained were left standing downward at 60° C. for one week and then used to evaluate a bubble discharge property according to the following evaluation criteria.

Evaluation Criteria:

◯: bubbles were produced

x: bubbles were not produced

Evaluation Method of Comprehensive Evaluation

The results of the evaluations obtained by the evaluation methods described above were subjected to comprehensive evaluation according to the following evaluation criteria.

Evaluation Criteria:

◯: up to one Δ

Δ: up to two Δ

x: three or more Δ or one x

TABLE 1
(Total: 100% by mass)

| | | Example | | | | | |
|---|---|---|---|---|---|---|---|
| | | 1 | 2 | 3 | 4 | 5 | 6 |
| Base Oil A | *1 | 93 | 92 | 93.5 | 93 | 92 | 96.5 |
| Base Oil B | *2 | | | | | | |
| Base Oil C | *3 | | | | | | |
| Elastomer A | *4 | 7 | 8 | 6.5 | | | |
| Elastomer B | *5 | | | | 7 | 8 | 3.5 |
| Colorant | *6 | | | | | | |

| | | | | | | |
|---|---|---|---|---|---|---|
| Hue of Follower | Colorless | Colorless | Colorless | Colorless | Colorless | Colorless |
| Specific Gravity to | 0.87 | 0.87 | 0.87 | 0.87 | 0.87 | 0.87 |
| Liquid Cosmetic | | | | | | |
| Average tan δ for | 0.265 | 0.259 | 0.332 | 0.261 | 0.264 | 0.279 |
| 1 to 20 Hz | | | | | | |
| (refer to Table 2 | | | | | | |
| for details) | | | | | | |
| Aging Stability | ∘ | ∘ | ∘ | ∘ | ∘ | ∘ |
| Usability | ∘ | ∘ | ∘ | ∘ | ∘ | ∘ |
| Followability | ∘ | ∘ | ∘ | ∘ | ∘ | ∘ |
| Dripping Resistance | ∘ | ∘ | ∘ | ∘ | ∘ | ∘ |
| Bubble Discharge | ∘ | ∘ | ∘ | ∘ | ∘ | ∘ |
| Property | | | | | | |
| Comprehensive | ∘ | ∘ | ∘ | ∘ | ∘ | ∘ |
| Evaluation | | | | | | |

| | | Comparative Example | | | | | |
|---|---|---|---|---|---|---|---|
| | | 1 | 2 | 3 | 4 | 5 | 6 |
| Base Oil A | *1 | 90 | 94 | 90 | 97 | | |
| Base Oil B | *2 | | | | | 93.5 | |
| Base Oil C | *3 | | | | | | 93.5 |
| Elastomer A | *4 | 10 | 6 | | | 3 | |
| Elastomer B | *5 | | | 10 | 3 | | 3 |
| Colorant | *6 | | | | | 3.5 | 3.5 |

| | | | | | | |
|---|---|---|---|---|---|---|
| Hue of Follower | Colorless | Colorless | Colorless | Colorless | Black | Black |
| Specific Gravity to | 0.87 | 0.87 | 0.87 | 0.87 | 0.87 | 0.87 |
| Liquid Cosmetic | | | | | | |
| Average tan δ for | 0.286 | 0.223 | 0.134 | 0.384 | 2.032 | 0.941 |
| 1 to 20 Hz | | | | | | |
| (refer to Table 2 | | | | | | |
| for details) | | | | | | |
| Aging Stability | ∘ | ∘ | ∘ | ∘ | Δ | Δ |
| Usability | x | ∘ | x | ∘ | ∘ | ∘ |
| Followability | x | ∘ | x | ∘ | ∘ | ∘ |
| Dripping Resistance | ∘ | x | ∘ | x | x | x |
| Bubble Discharge | x | ∘ | x | ∘ | ∘ | ∘ |
| Property | | | | | | |
| Comprehensive | x | x | x | x | x | x |
| Evaluation | | | | | | |

The terms *1 to *6 show the followings.
*1: Liquid paraffin, MORESCO WHITE P-350P (manufactured by MORESCO Corporation)
*2: Mineral oil, Diana Process Oil PW-380 (manufactured by Idemitsu Kosan Co., Ltd.)
*3: Squalane (manufactured by Kishimoto Special Liver Oil Co., Ltd.)
*4: Styrene elastomer G1701EU (styrene-ethylene/propylene block copolymer-(SEP, x:y = 37:63), manufactured by Kraton Polymer Japan Co., Ltd.)
*5: Styrene elastomer G1702HU (styrene-ethylene/propylene block copolymer-(SEP, x:y = 30:70), manufactured by Kraton Polymer Japan Co., Ltd.)
*6: oil-soluble dye, Oil Black 860 (manufactured by Orient Chemical Industries Co., Ltd.)

TABLE 2

| Respective tan δ for | | | | | | | | |
|---|---|---|---|---|---|---|---|---|
| 1 Hz (6.3 rad/sec) to | | | Example | | | | | |
| 20 Hz (125.6 rad/sec) | | | 1 | 2 | 3 | 4 | 5 | 6 |
| tan δ | 125.6 | rad/sec | 0.381 | 0.380 | 0.424 | 0.401 | 0.394 | 0.402 |
| tan δ | 111.2 | rad/sec | 0.369 | 0.368 | 0.414 | 0.385 | 0.380 | 0.392 |
| tan δ | 98.0 | rad/sec | 0.356 | 0.355 | 0.403 | 0.370 | 0.365 | 0.382 |
| tan δ | 86.7 | rad/sec | 0.343 | 0.342 | 0.393 | 0.354 | 0.351 | 0.369 |
| tan δ | 76.0 | rad/sec | 0.331 | 0.330 | 0.382 | 0.340 | 0.338 | 0.358 |
| tan δ | 67.2 | rad/sec | 0.318 | 0.318 | 0.372 | 0.325 | 0.325 | 0.345 |
| tan δ | 59.4 | rad/sec | 0.307 | 0.306 | 0.361 | 0.314 | 0.312 | 0.334 |
| tan δ | 52.5 | rad/sec | 0.297 | 0.296 | 0.352 | 0.299 | 0.301 | 0.322 |
| tan δ | 46.3 | rad/sec | 0.287 | 0.286 | 0.344 | 0.287 | 0.289 | 0.310 |
| tan δ | 40.8 | rad/sec | 0.277 | 0.275 | 0.336 | 0.276 | 0.279 | 0.299 |
| tan δ | 36.1 | rad/sec | 0.268 | 0.263 | 0.328 | 0.265 | 0.269 | 0.288 |
| tan δ | 31.9 | rad/sec | 0.260 | 0.255 | 0.321 | 0.254 | 0.260 | 0.278 |
| tan δ | 28.1 | rad/sec | 0.252 | 0.247 | 0.316 | 0.245 | 0.251 | 0.269 |
| tan δ | 24.8 | rad/sec | 0.245 | 0.239 | 0.310 | 0.236 | 0.243 | 0.259 |
| tan δ | 21.9 | rad/sec | 0.238 | 0.232 | 0.305 | 0.227 | 0.235 | 0.250 |
| tan δ | 19.4 | rad/sec | 0.232 | 0.225 | 0.302 | 0.220 | 0.228 | 0.242 |
| tan δ | 17.0 | rad/sec | 0.226 | 0.218 | 0.297 | 0.212 | 0.221 | 0.234 |
| tan δ | 15.1 | rad/sec | 0.221 | 0.213 | 0.297 | 0.206 | 0.213 | 0.226 |
| tan δ | 13.3 | rad/sec | 0.216 | 0.206 | 0.294 | 0.202 | 0.208 | 0.219 |
| tan δ | 11.7 | rad/sec | 0.211 | 0.200 | 0.292 | 0.197 | 0.201 | 0.213 |
| tan δ | 10.4 | rad/sec | 0.207 | 0.196 | 0.291 | 0.194 | 0.196 | 0.207 |
| tan δ | 9.1 | rad/sec | 0.201 | 0.194 | 0.292 | 0.189 | 0.191 | 0.201 |
| tan δ | 8.0 | rad/sec | 0.195 | 0.186 | 0.291 | 0.182 | 0.187 | 0.196 |
| tan δ | 7.1 | rad/sec | 0.196 | 0.178 | 0.292 | 0.175 | 0.179 | 0.190 |
| tan δ | 6.3 | rad/sec | 0.196 | 0.178 | 0.292 | 0.168 | 0.174 | 0.187 |
| Average | | | 0.265 | 0.259 | 0.332 | 0.261 | 0.264 | 0.279 |
| Respective tan δ for | | | | | | | | |
| 1 Hz (6.3 rad/sec) to | | | Comparative Example | | | | | |
| 20 Hz (125.6 rad/sec) | | | 1 | 2 | 3 | 4 | 5 | 6 |
| tan δ | 125.6 | rad/sec | 0.429 | 0.317 | 0.188 | 0.507 | 3.320 | 0.466 |
| tan δ | 111.2 | rad/sec | 0.413 | 0.310 | 0.183 | 0.496 | 3.260 | 0.475 |
| tan δ | 98.0 | rad/sec | 0.393 | 0.303 | 0.177 | 0.483 | 3.170 | 0.489 |
| tan δ | 86.7 | rad/sec | 0.379 | 0.294 | 0.171 | 0.469 | 2.550 | 0.499 |
| tan δ | 76.0 | rad/sec | 0.363 | 0.286 | 0.164 | 0.456 | 2.480 | 0.510 |
| tan δ | 67.2 | rad/sec | 0.348 | 0.277 | 0.158 | 0.444 | 2.460 | 0.527 |
| tan δ | 59.4 | rad/sec | 0.334 | 0.267 | 0.153 | 0.431 | 2.390 | 0.550 |
| tan δ | 52.5 | rad/sec | 0.321 | 0.258 | 0.148 | 0.418 | 2.330 | 0.576 |
| tan δ | 46.3 | rad/sec | 0.308 | 0.248 | 0.143 | 0.405 | 2.270 | 0.608 |
| tan δ | 40.8 | rad/sec | 0.297 | 0.239 | 0.139 | 0.393 | 2.230 | 0.646 |
| tan δ | 36.1 | rad/sec | 0.286 | 0.230 | 0.140 | 0.382 | 2.190 | 0.687 |
| tan δ | 31.9 | rad/sec | 0.276 | 0.221 | 0.128 | 0.371 | 2.100 | 0.734 |
| tan δ | 28.1 | rad/sec | 0.267 | 0.212 | 0.127 | 0.361 | 2.040 | 0.787 |
| tan δ | 24.8 | rad/sec | 0.258 | 0.204 | 0.124 | 0.354 | 1.980 | 0.845 |
| tan δ | 21.9 | rad/sec | 0.250 | 0.196 | 0.121 | 0.346 | 1.860 | 0.914 |
| tan δ | 19.4 | rad/sec | 0.243 | 0.189 | 0.119 | 0.341 | 1.790 | 0.986 |
| tan δ | 17.0 | rad/sec | 0.237 | 0.183 | 0.115 | 0.336 | 1.680 | 1.070 |
| tan δ | 15.1 | rad/sec | 0.232 | 0.177 | 0.113 | 0.331 | 1.620 | 1.160 |
| tan δ | 13.3 | rad/sec | 0.228 | 0.172 | 0.111 | 0.326 | 1.480 | 1.250 |
| tan δ | 11.7 | rad/sec | 0.223 | 0.167 | 0.108 | 0.324 | 1.430 | 1.350 |
| tan δ | 10.4 | rad/sec | 0.219 | 0.164 | 0.108 | 0.323 | 1.360 | 1.460 |
| tan δ | 9.1 | rad/sec | 0.217 | 0.163 | 0.106 | 0.322 | 1.300 | 1.560 |
| tan δ | 8.0 | rad/sec | 0.214 | 0.162 | 0.103 | 0.322 | 1.200 | 1.670 |
| tan δ | 7.1 | rad/sec | 0.212 | 0.164 | 0.102 | 0.325 | 1.150 | 1.780 |
| tan δ | 6.3 | rad/sec | 0.210 | 0.169 | 0.101 | 0.327 | 1.150 | 1.930 |

| | | | | | | |
|---|---|---|---|---|---|---|
| Average | 0.286 | 0.223 | 0.134 | 0.384 | 2.032 | 0.941 |

As apparent from the results shown in Table 1 and Table 2, it has been able to be confirmed that the cosmetic applicators prepared in Examples 1 to 6 falling in the scope of the present invention are excellent in an aging stability, a usability, a followability, a dripping resistance, a bubble discharge property, and comprehensive evaluation as compared with those prepared in Comparative Examples 1 and 6 falling outside the scope of the present invention.

### INDUSTRIAL APPLICABILITY

A follower composition for a cosmetic applicator by which a wet coating film can easily be applied on a nail and removed is obtained.

### EXPLANATION OF CODES

A, B: cosmetic applicators
10: filling chamber
20: liquid cosmetic
25: follower composition for a cosmetic applicator
30: coating part

1. A follower composition for a cosmetic applicator comprising at least a base oil and 3.5 to 8% by mass of an elastomer having a styrene structure.

2. The follower composition for a cosmetic applicator as described in claim 1, wherein the elastomer having a styrene structure comprises a styrene-ethylene/propylene block copolymer.

3. The follower composition for a cosmetic applicator as described in claim 1, wherein the base oil is a liquid paraffin.

4. The follower composition for a cosmetic applicator as described in claim 1, wherein an average value of tan δ in a frequency range of 1 to 20 Hz is 0.25 to 0.45.

5. The follower composition for a cosmetic applicator as described in claim 2, wherein the base oil is a liquid paraffin.

6. The follower composition for a cosmetic applicator as described in claim 2, wherein an average value of tan δ in a frequency range of 1 to 20 Hz is 0.25 to 0.45.

7. The follower composition for a cosmetic applicator as described in claim 3, wherein an average value of tan δ in a frequency range of 1 to 20 Hz is 0.25 to 0.45.

8. The follower composition for a cosmetic applicator as described in claim 5, wherein an average value of tan δ in a frequency range of 1 to 20 Hz is 0.25 to 0.45.
